(19) United States
(12) Patent Application Publication
(10) Pub. No.: US 2015/0135870 A1
(43) Pub. Date: May 21, 2015

(54) SURGICAL INSTRUMENT

(71) Applicants: Stephen KOSCHER, Spaichingen (DE); Johann WÜRTZ, (US)

(72) Inventors: Stephan Koscher, Spaichingen (DE); Johann Würtz, Spaichingen (DE)

(21) Appl. No.: 14/366,142

(22) PCT Filed: Dec. 7, 2012
(86) PCT No.: PCT/EP2012/074726

§ 371 Date: Jun. 17, 2014

(30) Foreign Application Priority Data
Dec. 19, 2011 (DE) 10 2011 056 596.5

(51) Int. Cl. A61B 17/00

(52) U.S. Cl. CPC A61B 17/00234; A61B 2019/4868
USPC 74/102

(57) ABSTRACT

In a surgical instrument (1), in particular a sliding shaft instrument, with a shaft (2), and with a slide (3) mounted slidably and/or releasably on the shaft (2), wherein the slide (3) is guided with at least one guide element (7,8) in at least one longitudinal groove (10,11) of the shaft (2), the surgical instrument (1) is to have at least one recess in the area of the longitudinal groove (10,11).

### BACKGROUND OF THE INVENTION

The invention relates to a surgical instrument.

The present invention is intended to relate to all possible types of surgical instruments, including for example to tubular shaft instruments. In particular, however, it relates to sliding shaft instruments. These are used to carry out cutting, shearing, clamping or similar operations, for example in the human body. In these operations, suitable handpieces are used to move the slide along a sliding surface of the shaft, and a jaw is generally actuated at the end thereof.

Sliding shaft instruments of this type are used primarily as a rongeur, bone punch, ear forceps and for gynecological biopsies. A general problem is that surgical instruments of this kind are products having a very wide variety of articulations, guides or grooves and channels. Such instruments are therefore extraordinarily difficult to clean and to sterilize, although cleaning of surgical instruments is of course of the utmost importance.

The object of the invention is to provide a surgical instrument, in particular a sliding shaft instrument, which can be more easily cleaned.

### SUMMARY OF THE INVENTION

This object is achieved by way of the features of the present invention, wherein a surgical instrument, in particular a sliding shaft instrument, comprises a shaft and a slide that is fastened to the shaft in a slidable and/or releasable manner. Preferably, the slide is guided by way of at least one guide element in at least one longitudinal groove in the shaft. Even more preferably, the shaft has at least one cutout in the region of the longitudinal groove. This produces the advantage that the longitudinal groove is easier to clean.

The expression “in the region of the longitudinal groove” means for the present invention that the cutout is arranged such that it opens the longitudinal groove at least to one side, such that the longitudinal groove is easier to clean and liquid which has penetrated into the longitudinal groove can pass more easily out of the longitudinal groove again.

In typical exemplary embodiments, the surgical instrument has a plurality of guide elements, a plurality of longitudinal grooves and a plurality of cutouts.

Expediently, the at least one cutout is recessed laterally into the shaft such that the longitudinal groove is opened. Preferably, the cutout is formed such that it passes right through the shaft and results in no grooves in which tissue residues or liquids can collect.

A further advantage of the cutout is that tissue residues or liquids which pass into the longitudinal groove can pass immediately out of the latter again and do not represent an obstacle for the guide element. As a result, jamming or stiffness of the shaft with respect to the slide is avoided.

In further exemplary embodiments, the at least one cutout is recessed on an underside and/or a top side of the shaft. Preferably, the cutout is recessed from the underside and/or the top side such that the longitudinal groove is opened from below. This likewise produces the advantage that liquid or tissue that passes into the longitudinal groove can pass back out of the latter in a simple manner.

In typical exemplary embodiments, a cutout from below and/or above and a lateral cutout are arranged in a region of a longitudinal groove.

The concept of the invention also comprises arranging the guide elements on the shaft. Preferably, the longitudinal grooves for guiding the guide elements are then recessed into the slide. The cutouts are then likewise recessed in the region of the longitudinal groove in the slide.

Preferably, the cutout is configured in the form of a slot. Preferably, the cutout has a rectangular, square, polygonal, round and/or crescent shape.

### BRIEF DESCRIPTION OF THE DRAWINGS

The invention is briefly described in the following text with reference to the appended drawings, in which:

FIG. 1 shows a side view of a dismantled sliding shaft instrument;

FIG. 2 shows a plan view of a shaft of the sliding shaft instrument according to FIG. 1;

FIG. 3 shows a side view of the sliding shaft instrument according to FIG. 1 in an assembled state.

### DETAILED DESCRIPTION

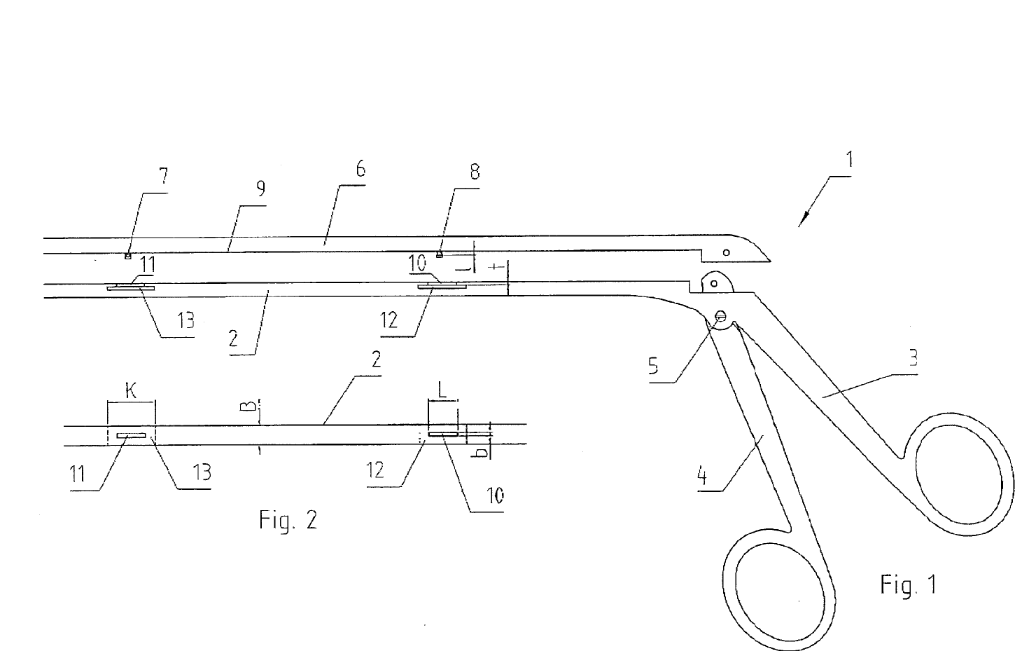

The surgical instrument shown in FIG. 1 is a sliding shaft instrument 1. The sliding shaft instrument 1 comprises a shaft 2 which merges integrally into a handle 3. Inserted between the shaft 2 and the handle 3 is a further handle 4. The further handle 4 is connected to the handle 3 in an articulated manner via an articulation 5.

The shaft 2 is assigned a slide 6. The slide 6 has two guide elements 7 and 8. In the present exemplary embodiment, the guide elements 7 and 8 are in the form of pins or T-shaped sliding blocks which project from an underside 9 of the slide 6.

The guide elements 7 and 8 have a length 1. The guide elements 7 and 8 are substantially in the form of pins or T-shaped sliding blocks and have preferably a diameter which corresponds substantially to the width b of the longitudinal grooves 10 and 11 such that the latter can be guided.

The shaft 2 comprises a first longitudinal groove 10 and a second longitudinal groove 11. The longitudinal grooves serve as counterparts for the guide elements 7 and 8 and are arranged such that the guide elements 7 and 8 can be guided in these longitudinal grooves 10 and 11.

The longitudinal grooves 10 and 11 have a depth t. The depth t is preferably less than a length 1 of the guide elements 7 and 8.

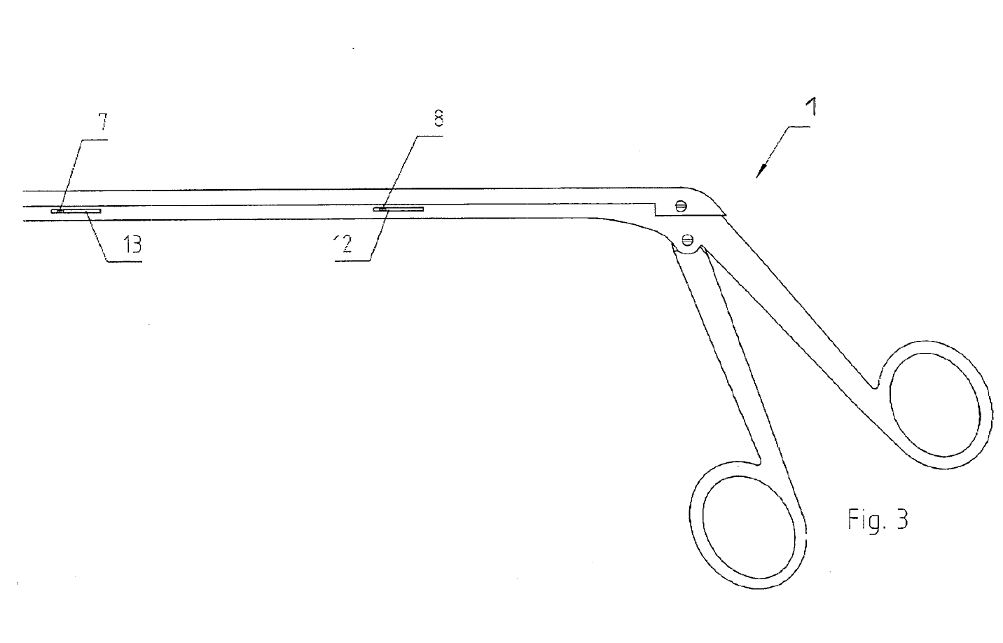

FIG. 2 shows a plan view of the shaft 2. As FIG. 2 shows, the longitudinal grooves 10 and 11 each have a width b and a length L.

Furthermore, the shaft 2 comprises a slot-like cutout 12 and 13 in each case in a region of the longitudinal grooves 10 and 11. As illustrated in FIG. 1, the cutouts 12 and 13 are recessed into the shaft 2 such that they open the longitudinal grooves 10 and 11.

Preferably, the cutouts 12 and 13 have a length K which is equal to or greater than a length L of the guide grooves 10 and 11.

Preferably, the cutouts 12 and 13, as indicated by way of dashed lines in FIG. 2, are recessed into the shaft 2 over an entire width B of the shaft 2. This produces the advantage that the guide elements 8 and 9 and the longitudinal grooves 10 and 11 (not illustrated in FIG. 2), can be cleaned more easily. Furthermore, soiling which is located between the guide elements 7 and 8 and the corresponding longitudinal grooves 11 and 10 can fall out through the cutouts 12 and 13 and thus does not impair the sliding movements between the shaft 2 and slide 3.

1-7. (canceled)

8. A sliding shaft surgical instrument, comprising a shaft and a slide fastened to the shaft in a slidable and/or releasable manner, wherein the slide is guided by way of at least one guide element provided in at least one longitudinal groove in the shaft, the surgical instrument has at least one cutout in the region of the longitudinal groove.

9. The surgical instrument as claimed in claim 8, wherein the at least one cutout is recessed into the shaft.

10. The surgical instrument as claimed in claim 8, wherein the at least one cutout is recessed into the slide.

11. The surgical instrument as claimed in claim 9, wherein the at least one cutout is recessed laterally such that the longitudinal groove is opened.

12. The surgical instrument as claimed in claim 11, wherein the at least one cutout is recessed from above and/or from below and/or laterally with respect to the longitudinal groove such that the longitudinal groove is opened.

13. The surgical instrument as claimed in claim 12, wherein the cutout is in the form of a slot.
